(19) United States
(12) Patent Application Publication
(10) Pub. No.: US 2006/0062744 A1
(43) Pub. Date: Mar. 23, 2006

(54) Composition for whitening teeth and method of making the same

(76) Inventor: Jeffrey Lokken, Buffalo, NY (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 11/227,004

(22) Filed: Sep. 15, 2005

Related U.S. Application Data
(60) Provisional application No. 60/611,009, filed on Sep. 17, 2004.

(51) Int. Cl. A61K 8/22

(52) U.S. Cl. 424/53

(57) ABSTRACT

A composition for whitening teeth being an anhydrous mixture of a polyol carrier material, a stabilized oxidizing agent, and a surfactant, the mixture having a viscosity sufficient to adhere to moistened tooth enamel while being substantially free of water soluble thickener, said anhydrous mixture being substantially shelf stable due to the absence of water, and wherein said mixture when applied to moistened tooth enamel oxidizes organic compounds without gel blocking A method for mixing such a composition is also shown.

### FIELD OF THE INVENTION

This invention relates generally to the field of oral care products, and more particularly to oral care products that whiten teeth.

### BACKGROUND OF THE INVENTION

Tooth whitening products are well known and are used to lighten the color of teeth for aesthetic reasons. Many forms of whitening agents or actives are used and are delivered in many types of products. Tooth whitening actives are used in toothpastes or dentifrices, gels, both paint on and tray-based systems, gums and strips, including strips having backing layers, among other things.

A problem for such tooth whitening products is to provide a product that, on the one hand has a long shelf life (and thus remains active and effective for a considerable time) and one which is faster acting, so the whitening step can be accomplished quickly without inconvenience to the user. The more reactive the tooth whitening active the quicker the tooth whitening occurs. Of course, a more reactive tooth whitening formulation is typically less stable. Thus, more stable whitening compositions tend to be slow acting, while faster acting compositions tend to be less stable. What is desired is a tooth whitening composition having a tooth whitening active which is both fast acting and relatively shelf stable over reasonable shelf storage times.

Typically, tooth-whitening compositions will include a form of tooth whitening active, such as a hydrogen peroxide or a hydrogen peroxide precursor such as carbamide peroxide. When introduced to water, the precursor dissociates into urea and hydrogen peroxide. Typically one or more polymers or other substances are used in a viscous carrier such as glycerin or glycol to prevent premature dissociation. Water typically forms a part of the composition and thickeners are used to control viscosity and to prevent the water from separating out of the composition. The use of such thickeners to help form the viscous gel leads to other problems for the composition. For example, the use of such thickeners prevents the active from migrating to the tooth surface. This is referred to as gel blocking. Where the tooth whitening composition is used in a composition that is brushed onto the teeth, such as a dentifrice, this is not so much of a concern since the act of brushing will cause fresh active to be introduced to the tooth surface. For paint on gels, tray-based systems and aqueous-based strips, the inability of the active to move through the thickened gel is a serious limitation.

U.S. Pat. Nos. 5,614,174, 5,690,913 and 5,599,525 to Colgate Palmolive Company disclose a dentifrice with up to 9% water content which includes either Xanthan or Sylodent 15, as a thickening agent. The use of these compositions will both thicken the gel and make it more difficult for the active to migrate through the gel to reach the tooth surface. However, for a dentifrice which is applied in the normal course by tooth brushing, lack of movement of the active through the thickened gel is not a problem due to physical mixing and remixing of the composition and a spreading of the composition onto the tooth surface introducing fresh active during such brushing.

U.S. Pat. No. 6,221,341 proposes another form of tooth whitening agent, in which two separate chemicals are brought together, in the act of applying the composition in the mouth which, through their interaction, create the tooth whitening active. In the preferred example the combination of a hydrogen peroxide precursor with glyceryl triacetate and water creates peroxyacetic acid as the tooth whitening active. However, this requires separate compartments in the dispensing container to keep the components apart during storage and a form of dispenser that ensures good mixing as the material leaves the container to ensure the appropriate conditions occur to create the active. This requires a more complex and expensive form of container and compounds packaging costs.

U.S. Pat. Nos. 6,682,721 and 6,689,344 disclose a dry type tooth whitening patch having peroxide as a tooth whitening agent. The patch is a hydrophilic glass polymer that has a strong adhesion to the tooth. However, a patch system is less convenient than a paint on gel.

What is desired is a tooth whitening composition that is both fast acting (in a mater of minutes rather than hours) and yet which discloses a high stability and long shelf life. Preferably such a composition includes a stable hydrogen peroxide precursor, which is not likely to dissociate before being placed in the mouth.

### SUMMARY OF THE INVENTION

The present invention is directed, in one aspect, to a relative thick or viscous anhydrous formulation for application directly to teeth to whiten the same. The anhydrous formulation is preferably comprised of a polyol carrier material such as glycerin into which a stabilized oxidizing agent such as PVP hydrogen peroxide is mixed. Most preferably the PVP hydrogen peroxide is cross linked which adds viscosity renders the PVP substantially insoluble in water and yet provides a stable oxidizing agent which remains reactive over time due to both its stability and the absence of water in the formulation. The cross-linked PVP according to the present invention shows good mixing with the polyol. The cross-linked PVP does not solubilize with the anhydrous polyol media. A surfactant is also added to lower the surface tension of the water on the tooth surface when the formulations of the present invention becomes hydrated from the saliva on the tooth surface. This will lower the surface tension and will further increase the migration of active to the tooth surface. The formulation according to the present invention demonstrates reasonable shelf life, strong tooth whitening and rapid action, meaning that the oxidizing agent is able to come to bear on the surfaces to be whitened in commercially effective concentrations over the course of a short, generally less than ten minute and most preferably about five minute, treatment.

Most preferable the cross-linked PVP hydrogen peroxide will be sufficiently viscous so as to, in combination with the other ingredients, cause the formulation to adhere to the teeth of a user.

Therefore according to a first aspect of the invention there is provided a composition for whitening teeth comprising:

an anhydrous mixture of a polyol carrier material, a stabilized oxidizing agent, and a surfactant, said mixture having a viscosity sufficient to adhere to moistened tooth enamel while being substantially free of water soluble thickener, said anhydrous mixture being substantially shelf stable due to the absence of water, and wherein said mixture when applied to said moistened tooth enamel oxidizes organic compounds without gel blocking.

According to a further aspect of the present invention there is disclosed a method of whitening teeth comprising applying a formulation comprised of an anhydrous mixture of a stabilized oxidizing agent, a polyol carrier material and a surfactant to a tooth surface to be whitened, letting the formulation remain on the teeth for an effective tooth whitening period wherein the tooth whitening period is between four and six minutes.

According to a further aspect of the present invention there is provided a composition for whitening teeth comprising an anhydrous mixture of a polyol carrier material and an oxidizing agent, wherein said oxidizing agent does not substantially dissociate in said anhydrous mixture until said mixture is applied to a moistened tooth surface.

### DETAILED DESCRIPTION OF THE PREFERRED EMBODIMENTS

The formulation made according to the present invention is an anhydrous mixture of a polyol carrier material, a stabilized active and a surfactant. Other additives may also be used to improve the characteristics of the formulation such as colorants, sweeteners, flavoring agents, accelerants and the like, as discussed in more detail below. In this specification the terms formulation and composition are used interchangeably.

Turning first to the polyol, the preferred polyol is glycerin. Although glycerin is referred to commercially as being anhydrous, in fact it may contain trace amounts of water, typically less than 0.5%. Being a humectant though it has an affinity for water and can absorb water from air, for example during manufacturing. The present invention can tolerate small amounts of water and for the purposes of this disclosure the term anhydrous is intended to mean a water content of less than about 2% by weight of the overall solution. Essentially the present invention requires that there so little water present that the gel is not characterized as an aqueous gel nor is an aqueous thickener required.

While the most preferred polyol is glycerin other polyols can also be reasonably substituted. These include anhydrous or relatively anhydrous polyols. Polyols may be defined as hydrocarbons, commonly from carbohydrates, that have had some of their carbonyl groups reduced to a primary or secondary hydroxyl group(s) and be more simply defined an organic compound having more than one hydroxyl (—OH) group per molecule and alcohols. Examples of such polyols are glycerin, propylene glycol, butylene glycol, and propylene glycol, and an alcohol, such as 200 proof anhydrous ethanol.

The polyol is included in the formulation of the present invention at a concentration of about 10% to about 85% by weight.

Turning now to the whitening active, the most preferred form of whitening active is a cross-linked polyvinylpyrrolidone hydrogen peroxide (PVP-H2O). The cross-linking renders the material mostly water insoluble, which helps to stabilize the mixture in the anhydrous formulation even the presence of trace amounts of water. Other actives include uncrosslinked anhydrous Polyvinylpyrrolidone hydrogen peroxide. This may in some case be suitable but is less preferred for two reasons. Firstly, it is less stable and so is more likely to have a reduced shelf life and secondly, it tends to be less viscous meaning that it is harder to achieve the desired viscosity with the uncross linked PVP H2O.

The active is included in the formulation of the present invention at a concentration of about 10 to 60% by weight. Most preferably the active has a molecular weight of about 15 to 50. As will be appreciated by those skilled in the art, about 20% (w/w) of PVPH202 is hydrogen peroxide, so for example 15% to 50% PVP-H202 range is actually 3% to 10% H2O2. The percentage of PVP-H202 as measured by concentration of H2O2 is between 1 and 15% (w/w) and most preferably between 1 and 10% (w/w).

Surfactants are typically added reduce the surface tension. In aqueous gel formulations of the prior art the presence of significant concentrations of water results in a thin or fluid composition that requires a thickener to achieve a workable viscosity. Low viscosity is to be avoided because it affects the control over the location of the whitening product in the mouth. For example if the gel is too runny the active will run off the teeth, may spill out of a tray or otherwise becomes mobile and thus is more likely to come into contact with sensitive gingival tissues. As a result since surfactants reduce viscosity, aqueous based systems do not include any surfactants.

According to the present invention surfactants are desirable to reduce the viscosity of the anhydrous formulation of a mixture of stabilized oxidizing agent and polyol that makes it easier to apply to the tooth surface, encourages better mixing of the ingredients and permits the active to more easily reach and therefore interact more quickly with organics located on the tooth surface. After the formulation of the present invention comes in contact with the saliva it will lower the surface tension to further increase the migration of active material in the invention. A lower viscosity permits a better tooth whitening rate and/or efficacy of the product via an increase in the migration of active ingredient to the tooth surface.

Examples of suitable surfactants according to the present invention are sodium dodecanyl sulfate (sodium lauryl sulfate) and sodium lauroyl sarcosinate. According to the present invention the surfactant is included in the formulation in an amount of about 0.1 to 5% by weight.

The present invention also provides for the optional addition of organoleptic ingredients. These ingredients contribute to the visual appeal, taste and odor of a product. Examples of Organoleptic ingredients according to the present invention include flavorants, colorants, pigments, dyes and sweeteners. Examples of sweeteners are sucralose, sodium saccharin, potassium acesulfame. Examples of dyes are those approved under the Food and Drug Cosmetics Act, and are under the category of FD&C and D&C dyes (Food Drug and Cosmetic). Examples are FD&C Red #4, FD&C Blue 1 and FD&C Yellow 5. Pigments and colorants may add color and/or opacity to a product. Examples of pigments and colorants are titanium dioxide, mica and the lake equivalents of FD&C and D&C dyes.

Some common flavorants that may add both taste and odor to the invention may include peppermint oil, spearmint oil, methyl salicylate and menthol. According to the present invention the preferred formulation is anhydrous, meaning the pH cannot be measured. However, when the formulation comes in contact with the saliva on the tooth surface, then the resulting mixture will have a certain pH. Depending upon the other ingredients, it may be advisable to add one or more pH-adjusting agents to the formulation to ensure that the tooth whitening formulation has an optimal pH when moistened by saliva when placed on teeth in the mouth. As well an alkaline agent can encourage the oxidation reaction so in one embodiment the present invention comprehends adding alkaline agent such as Triethanolamine. The alkaline agent is preferably in a small amount of less than 1% by weight and most preferably about 0.5% by weight of the total formulation.

While the primary embodiment of the present invention is as a viscous formula to be applied directly to the teeth, in another aspect it can be used as a foundation to a dentifrice formulation. In such an embodiment it is preferred to add to the formulation silicas. Silicas and hydrated silicas are added to act as abrasive agents and rheological modifiers. Also, it can be used in a less viscous formulation in a pen-based or brush on application.

Inorganic polyphosphate salts may also be added as anti-tartar agents, such as sodium tripolyphosphate, sodium hexametaphosphate, tetrapotassium pyrophosphate and tetrasodium pyrophosphate.

To prepare the formulations of the present invention the polyol i.e. glycerin, the surfactant and the whitening active are dispersed in a conventional mixer until the mixture becomes a homogeneous phase. Then, into the homogeneous phase are added the remaining ingredients such as flavoring or other organoleptic agents, anti tartar agents, and the like. Thereafter the ingredients are further mixed until a homogenous consistency is achieved. The desired viscosity is between 50,000 and 1,500,000 centipoises and more preferably between 300,000 and 1,200,000.

It will be understood by those skilled in the art that because the formulations of the present invention are anhydrous, care must be taken during manufacture to limit the exposure to potential sources of water, such as humid air. As well, if the viscosity needs to be changed to achieve the desired viscosity, this must be done without using water or thickeners as is traditionally been the case for these types of compositions.

### EXAMPLE I

Table I below details various embodiments of the present made following the above-described standard procedure. These embodiments are depicted as formulations A to F on the Table. Table II below depicts alternate embodiments of the present invention using PVP-H2O which is not cross-linked. The present invention also comprehends mixtures of cross-linked and uncross-linked PVP-H202 being combined with the anhydrous polyol media, to combine lower viscosity with improved stability. The method of preparation is generally the same, and different formulations of this embodiment are shown as A to D in the table. Table III below is a further embodiment of the present invention in which the viscous formulation is used as a base for a dentifrice. In this embodiment various other ingredients are added such as silica in the amounts shown, which also act to thicken the formulation.

TABLE I
Crosslinked PVP-H2O2 Formulations

| Ingredient | A | B | C | D | E | F |
|---|---|---|---|---|---|---|
| Crosslinked PVP-H2O2 | 21.0 | 21.0 | 21.0 | 21.0 | 21.0 | 30.0 |
| Glycerin | 62.7 | 63.2 | 30.0 | 30.2 | 30.7 | 54.0 |
| PEG-6 | | | 30.2 | 15.0 | 15.0 | |
| Propylene Glycol | | | | 15.0 | 15.0 | |
| Etidronic Acid | 0.5 | | | | | |
| Ethanol (200 Proof) | 15.0 | 15.0 | 18.0 | 18.0 | 18.0 | 15.0 |
| Alkaline Agent (e.g. | 0.5 | 0.5 | 0.5 | 0.5 | | 0.5 |
| Triethanolamine) | | | | | | |
| Surfactant (e.g. Sodium | 0.3 | 0.3 | 0.3 | 0.3 | 0.3 | 0.5 |
| Lauroyl Sarcosinate) | | | | | | |
| Total | 100.0 | 100.0 | 100.0 | 100.0 | 100.0 | 100.0 |

TABLE II
PVP-H2O2 Formulations

| Ingredient | A | B | C | D |
|---|---|---|---|---|
| PVP-H2O2 | 21.0 | 32.5 | 21.0 | 21.0 |
| Glycerin | 62.7 | 66.2 | | 63.0 |
| PEG-6 | | | 62.7 | |
| Ethanol | 15.0 | | 15.0 | 15.0 |
| Phosphoric Acid | | 0.5 | | |
| Etidronic Acid | 0.5 | | 0.5 | 0.5 |
| Alkaline Agent (e.g. Triethanolamine) | 0.5 | 0.5 | 0.5 | 0.5 |
| Surfactant (e.g. Sodium Lauroyl Sarcosinate) | 0.3 | 0.3 | 0.3 | |
| Total | 100.0 | 100.0 | 100.0 | 100.0 |

TABLE III
Crosslinked PVP-H2O2 Dentifrice Formulations

| | A | B | C | D |
|---|---|---|---|---|
| Crosslinked PVP-H2O2 | 3.00 | 5.00 | 5.00 | 5.00 |
| Glycerin | 64.77 | 53.60 | 54.00 | 56.00 |
| Xylitol | | | | 4.00 |
| PEG-18 | | 10.00 | 10.00 | 10.00 |
| Sodium Saccharin | 0.40 | 0.40 | | |
| Flavor | 0.50 | 0.50 | 0.50 | 0.50 |
| Titanium Dioxide | 1.50 | 1.50 | 1.50 | 1.50 |
| Sodium Tripolyphosphate | 3.00 | 3.00 | | |
| Sodium Monofluorophosphate | 0.83 | | | |
| Surfactant (e.g. Sodium Lauroyl | 1.00 | 1.00 | 1.00 | 1.00 |
| Sarcosinate) | | | | |
| Abrasive Silica (Hydrated Silica | 20.00 | 20.00 | 25.00 | 15.00 |
| e.g. Sylodent XWA 350) | | | | |
| Thickening Silica (Hydrated Silica | 5.00 | 5.00 | 3.00 | 7.00 |
| e.g. Zeodent 165) | | | | |
| Total | 100.00 | 100.00 | 100.00 | 100.00 |

Notes:
Sylodent XWA 350 is made by Grace Davison
Zeodent 165 made by J. M. Huber Corporation
stability further increased with the use of synthetic glycerin

### EXAMPLE II

An experiment was conducted to test the whitening efficacy and to test the safety of the formulation according to Table I of the present invention. The test conducted compared the effect of the present invention to other whitening products and one control product (i.e. placebo). The test was conducted on a study population of 200 subjects, male and female, who met predetermined inclusion and exclusion criteria. Eight groups were identified with twenty-five subjects in each group. The test was conducted on a single blind basis to optimize objectivity. Clinical measurements were taken at the start, after three days for certain groups and after two weeks for all groups. The clinical measurements included, an oral soft tissue exam, a tooth shade exam, a modified gingival index exam, a sensitivity questionnaire, and intraoral photographs were taken for spectrophotometric analysis for changes in shade.

In this test two different groups were given variations of the Table I present invention. In one group the subjects were to brush their teeth normally with regular toothpaste and a regular soft bristled brush. Then they placed a bead of formulation according to the present invention into upper and lower customized mouth trays that were then placed in the mouth. Contact of the formulation with the gums was to be avoided. The trays were left in place for five minutes then removed and excess gel was brushed off with tap water. The teeth whitening regimen was repeated twice per day. In one group the teeth were also thoroughly brushed at least twice per day. For the other group, everything was the same except they did not also brush their teeth twice per day.

In the case of the one group the results of two specific teeth for each subject that were measured at a baseline, and after two weeks, showed a starting whiteness of 15.1±−3.1 and a finishing whiteness of 12.0±3.3 for an average percentage increase of 21.8%±11.7%. For the other group, the starting whiteness was measured at 14.0±5.0 and after two weeks was measured at 9.7±5.9 for a percentage increase of 35.0%±23.4%. Brushing immediately after application of the formulation seems to reduce the effectiveness of the whitening treatment.

The conclusions drawn from the study are that the whitening obtained with the present invention is similar to those of other prior art formulations, and in particular to Crest White Strips( which showed an average increase of 42.3%±22.4%), when following the protocols of the study, except that instead of requiring two thirty minute application periods per day as does Crest White Strips in a two week treatment program the present invention requires two five minute application periods per day. The study further showed whitening occurred sooner in the present invention and the data showed the formulation was safe to oral tissues.

In summary the present invention demonstrates effective whitening in a short time due to the elimination of gel blocking which is typical for prior art compositions which use water and thickeners to achieve the desired viscosity. By providing an anhydrous formulation, in which the viscosity is achieved by mixing the active with a polyol media, and which coats the tooth surface well, through the help of a surfactant, the present invention provides a tooth whitening composition which demonstrates effective tooth whitening in short treatment session of less than ten minutes and most preferably about five minutes at a time. Further the use of a cross linked oxidizing agent which has both good viscosity and demonstrates improved stability provides an easy to use formulation which has a long stable shelf life.

I claim:

1. A composition for whitening teeth comprising:
an anhydrous mixture of a polyol carrier material, a stabilized oxidizing agent, and a surfactant, said mixture having a viscosity sufficient to adhere to moistened tooth enamel while being substantially free of water soluble thickener, said anhydrous mixture being substantially shelf stable due to the absence of water, and wherein said mixture when applied to said moistened tooth enamel oxidizes organic compounds without gel blocking.

2. A composition as claimed in claim 1 further including an alkaline agent to accelerate the oxidation of organic compounds upon the mixture being placed in contact with the moistened tooth enamel.

3. A composition as claimed in claim 1 wherein said stabilized oxidizing agent is a water insoluble polymer.

4. A composition as claimed in claim 2 wherein said water insoluble polymer is a cross linked PVP-H2O2.

5. A composition as claimed in claim 1 further including an organoleptic additive.

6. A composition as claimed in claim 5 further includes one or more of a flavorant or a colorant.

7. A composition for whitening teeth comprising:
an anhydrous mixture of a polyol carrier material and an oxidizing agent, wherein said oxidizing agent does not substantially dissociate in said anhydrous mixture until said mixture is applied to a moistened tooth surface.

8. The composition of claim 7 wherein the oxidizing agent is one or both of cross-link PVP-H202 and uncross-linked PVP-H202.

9. The composition of claim 8 further including an organoleptic additive.

10. The composition of claim 9 further including an accelerant.

11. The composition of claim 10 wherein said composition has a viscosity of between 50,000 and 1,500,000 centipoises.

12. The composition of claim 10 wherein said composition has a viscosity of between 300,000 and 1,200,000 centipoises.

13. The composition of claim 7 wherein the oxidizing agent is present in a range of 10% to 60% by weight.

14. The composition of claim 7 wherein the polyol is one or more glycerin, GEG-6, propylene glycol and ethanol.

15. The composition of claim 14 wherein the polyol is present in an amount of about 10% to 85% by weight.

16. A method of formulating a tooth whitening composition comprising:
starting with an anhydrous polyol carrier material;
mixing in about 10% to 60% by weight of an organic whitening active, wherein the active does not become substantially dissociated;
adding in a surfactant in an amount of between 0.1% and 5% by weight; and
mixing the ingredients until a homogeneous mixture is obtained of between 50,000 and 1,500,000 centipoises.
